(19) United States
(12) Patent Application Publication
(10) Pub. No.: US 2026/0128126 A1
(43) Pub. Date: May 7, 2026

(54) ITERATIVE ARTIFICIAL INTELLIGENCE METHODS AND SYSTEMS FOR DESIGN OPTIMIZATION IN SYNTHETIC BIOLOGY

(71) Applicant: Avery Bio Corporation, La Jolla, CA (US)

(72) Inventors: Barry Merriman, La Jolla, CA (US); Ryan de Ridder, San Diego, CA (US)

(21) Appl. No.: 19/380,340

(22) Filed: Nov. 5, 2025

Related U.S. Application Data
(60) Provisional application No. 63/716,330, filed on Nov. 5, 2024.

(51) Int. Cl. G16B 30/00; G06F 30/27; G16B 40/20

(52) U.S. Cl. CPC G16B 30/00; G06F 30/27; G16B 40/20

(57) ABSTRACT

A system for artificial intelligence (AI)-assisted production of an optimal design in synthetic biology is disclosed. The system includes a high-throughput experimental device; a generative AI; and experimental designs, in which a first set of designs of the experimental designs are configured to be tested and scored by the high-throughput experimental device and used for training the generative AI for production of a new set of designs by the generative AI. The system can include functional scoring assays and a problem compiler.

### CROSS-REFERENCE TO RELATED APPLICATION

This Application claims priority to U.S. Provisional Patent Application No. 63/716,330, filed Nov. 5, 2024, entitled “ITERATIVE ARTIFICIAL INTELLIGENCE METHODS AND SYSTEMS FOR DESIGN OPTIMIZATION IN SYNTHETIC BIOLOGY”, which is incorporated by reference in its entirety for all purposes.

### FIELD

This disclosure is in the field of synthetic biology. This disclosure is also in the field of artificial intelligence (“AI”). In particular, this disclosure describes systems and methods that are useful for optimizing a design in synthetic biology in order to achieve a desired biological function or outcome, or to maximize or minimize an assay result or metric, where such designs may comprise nucleic acid sequences, and the parameters that define the supporting cellular environments or processes.

### BACKGROUND

The field of synthetic biology is broadly concerned with the engineering biological processes to achieve desired functional endpoints. An area of focus is in designing and synthesizing the DNA sequence of cellular organisms as a means of engineering their functional properties.

### SUMMARY

In an aspect, a method and system for AI-assisted optimal design in synthetic biology is provided. In particular, a high-throughput experimental device is provided, along with a generative AI, which generates designs and learns from experimental testing of designs. A problem specification, experimental design, and scoring assays are also provided. A first set of designs are tested and scored using the experimental device. In embodiments, the first set of designs may be given, or generated by the AI, or any combination of these, plus optional expansion of such a variation set by random variation methods. Generative AI trains on this and generates new designs. Additionally, the process is iterated until the optimization criteria or termination conditions are achieved.

In another aspect, a method of optimizing design in synthetic biology by utilizing assistance of AI is provided. The method comprises receiving a first set of designs, wherein the first set of designs have been tested and scored using a high-throughput experimental device. The method also comprises training a generative AI system using the first set of designs. The method also comprises generating new designs using the generative AI system. Additionally, the method comprises iterating steps of testing and scoring designs, training a generative AI system based on the tested and scored designs, and generating a new set of designs using the generative AI system until at least one of optimization criteria or termination conditions are achieved.

In certain embodiments, the high-throughput experimental device is a semiconductor chip-based device. In certain embodiments, a method and system for fully autonomous optimal design in synthetic biology is provided and is combined with a problem compiler, in which a high-level problem and solution budget are provided, and where the problem compiler translates these into the initiation and control parameters of the system, and this iterates until stopping criteria are met. In certain embodiments, the AI is a generative AI based on deep learning neural networks and/or diffusion methods. In some embodiments, the AI algorithm is based on selection of a top performing fraction of designs, combined with random variation expansion of the designs, and using methods selection and variation methods from genetic programming algorithms, and/or simulated annealing algorithms, and/or Monte Carlo sampling algorithms. In some embodiments, the methods and/or systems provided herein are used for delivering biological DNA, protein, or cellular living materials representing optimal synthetic biology designs.

This disclosure outlines methods and systems for using AI algorithms to optimize designs in synthetic biology.

This disclosure outlines a method in which an AI algorithm can be put into an iterative context, in conjunction with a high throughout experimental device, in order to achieve a system and method for determining an optimal design in synthetic biology.

This disclosure outlines a method in which generative AI algorithms that were devised for basic design generation problems, can be put into an iterative context, in conjunction with a high throughout experimental device, in order to achieve a system and method for determining optimal designs in synthetic biology.

This disclosure outlines a method for and system for determining optimal designs in synthetic biology, based on a class of simple iterative algorithms coupled to a high throughout experimental device.

This disclosure outlines a system and method for the autonomous design optimization in synthetic biology, in which the inputs would be a desired function or desired parameter to maximize or minimize, or a vector of such parameters that is desired to lie in a specified target region of the parameter space, and which would then under autonomous action, achieve a design or designs that realize this goal.

This disclosure outlines methods and systems as detailed herein, where the high throughout experimental device is based on electronic semiconductor chip devices that carry out the end-to-end-workflow of DNA synthesis, assembly, cell packaging, and functional testing.

In aspects of the present disclosure, a method and system for AI-assisted optimal design in synthetic biology is disclosed. The method and system includes a high-throughput experimental device; a generative AI; a problem specification, experimental design, and scoring assays, in which a first set of designs (which may be given, generated by the AI, or any combination of these, plus optional expansion of such a variation set by random variation methods) tested and scored using the high-throughput experimental device, the generative AI trains on these elements and generates new designs, and the process is iterated until the optimization criteria or termination conditions are achieved.

In embodiments, the high-throughput experimental device is a semiconductor chip-based device.

In embodiments, the method and system is combined with a problem compiler, in which a high-level problem and solution budget are provided, and the problem compiler translates these into the initiation and control parameters of the system, and this iterates until certain stopping criteria are met.

In embodiments, the AI is a generative AI. In embodiments, the generative AI is based on deep learning neural networks or diffusion methods.

In embodiments, the AI algorithm is based on selection of a top performing fraction of designs, combined with random variation expansion of the designs, and using methods selection and variation methods selected from genetic programming algorithms, and/or simulated annealing algorithms, and/or Monte Carlo sampling algorithms.

In embodiments, the method and system is optimized to deliver biological DNA, protein, or cellular living materials representing optimal synthetic biology designs.

In another aspect, a method of optimizing design in synthetic biology by utilizing assistance of AI is disclosed. The method involves receiving a first set of designs, wherein the first set of designs have been tested and scored using a high-throughput experimental device; training a generative AI system using the first set of designs; generating new designs using the generative AI system; and iterating steps of testing and scoring designs; training a generative AI system based on the tested and scored designs; and generating a new set of designs using the generative AI system until at least one of optimization criteria or termination conditions are achieved.

In embodiments, the high-throughput experimental device is a semiconductor chip-based device.

In embodiments, the method involves providing a problem compiler having a high-level problem and a solution budget, and using the program compiler, translating the high-level problem and the solution budget into initiating and control parameters used in the iteration steps, and iterating until stopping criteria are met.

In embodiments, the AI is a generative AI. In embodiments, the generative AI is based on deep learning neural networks or diffusion methods.

In embodiments, the AI algorithm is based on selection of a top performing fraction of designs, combined with random variation expansion of the designs, and using methods selection and variation methods selected from genetic programming algorithms, and/or simulated annealing algorithms, and/or Monte Carlo sampling algorithms.

In embodiments, the method and system is optimized to deliver biological DNA, protein, or cellular living materials representing optimal synthetic biology designs.

In aspects of the present disclosure, a system for artificial intelligence (AI)-assisted production of an optimal design in synthetic biology is provided. In embodiments, the system comprises a high-throughput experimental device; a generative AI; and experimental designs; wherein a first set of designs of the experimental designs are configured to be tested and scored by the high-throughput experimental device and used for training the generative AI for production of a new set of designs by the generative AI.

In embodiments, the system further comprises functional scoring assays configured to constitute trial results for testing the first set of designs.

In embodiments, the system further comprises a problem compiler configured to be provided with a high-level problem statement, and a solution budget; and wherein the problem compiler is configured to use the high-level problem statement and the solution budget to compile initial specification and iteration control parameters for the system.

In embodiments, the production of a new set of designs is iterated until achieving optimization criteria or termination conditions.

In embodiments, the high-throughput experimental device is a semiconductor chip-based device. In some such embodiments, the semiconductor chip-based device comprises any one or more of a cell packaging and assay testing chip, a DNA assembly chip, and a DNA synthesis chip.

In embodiments, a biological representative of the optimal design comprises: DNA sequences, protein amino acid sequences, cell types to contain DNA, cell types to contain proteins, organisms, culture media, environmental conditions, treatment processes for exposure to nutrients or chemical signals, and cell-free expressions. In some such embodiments, the treatment processes comprise exposure to nutrients or chemical signals in a certain order, duration and dose, or growth to a level of multiple cells, cell populations, or organoids.

In embodiments, the generative AI is based on deep learning neural networks, or diffusion methods.

In embodiments, outputs of the system comprise biological constructs. In some such embodiments, the system further comprises a delivery system of the biological constructs.

In aspects of the present disclosure, a method of determining an optimal design for synthetic biology using generative AI is provided. In embodiments, the method comprises providing initial design parameters and iteration control parameters to a high-throughput experimental device; providing an initial set of trial designs; producing a set of design trial results; training a generative AI system using the set of design trial results; and generating a new design using the generative AI system. In some such embodiments, the method further comprises further training the generative AI system on the new design; and producing further sets of new designs; and wherein the steps of further training and producing further sets of new designs are an iterative AI method. In some such embodiments, the iterative AI method is repeated until a termination condition is achieved. In some such embodiments, the termination condition comprises a performance vector of a subset of produced designs achieving a predetermined target, reaching a target threshold, and a rate of increase or decrease of the initial design parameters and iteration control parameters undergoing optimization reaching a specified threshold.

In embodiments, the method using generative AI is fully autonomous.

In embodiments, the high-throughput experimental device is a semiconductor chip-based device.

In embodiments, the method further comprises producing a biological representative of the determined optimal design. In some such embodiments, the biological representative comprises: DNA sequences, protein amino acid sequences, cell types to contain DNA, cell types to contain proteins, organisms, culture media, environmental conditions, treatment processes for exposure to nutrients or chemical signals, and cell-free expressions.

In embodiments, an algorithm of the generative AI is based on one or more of: selection of a top performing fraction of the design trial results combined with random variation expansion of the design trial results, and methods selection and variation methods from genetic programming algorithms, methods selection and variation methods from simulated annealing algorithms, and methods selection and variation methods from Monte Carlo sampling algorithms.

### BRIEF DESCRIPTION OF THE DRAWINGS

FIG. 1 shows a general schematic of an iterative AI system for the optimization of a design in synthetic biology.

FIG. 2 shows a general schematic of an iterative AI system for the optimization of a design in synthetic biology, in which the high throughput experimental device is based on electronic semiconductor chips.

FIG. 3 shows a general schematic of iterative AI system for the fully autonomous optimization of a design problem in synthetic biology.

### DETAILED DESCRIPTION

This disclosure provides devices, methods, and systems related to using an AI that generates candidate synthetic biology designs, within an iteration experimental system and method that provides for optimal experimental solution of the design problem. This disclosure also covers compositions developed from such methods and systems.

Design Problems in Synthetic Biology. The field of synthetic biology is broadly concerned with the engineering biological processes to achieve desired functional endpoints. One major area of focus is in designing and synthesizing the DNA sequence of cellular organisms as a means of engineering their functional properties. In particular, this includes the need to design the relevant DNA sequence that correspond to individual genes, sets of genes, or larger genomic DNA constructs such as plasmids or chromosomal elements, up to the extremes of entire genomes of artificial organisms, and to provide such DNA payloads into the desired cells or cell-free environments, and also to determine the other related process variables, such as environmental conditions, concentrations of nutrients, growth factors other factors, and treatment procedures that cause such cells to carry out a desired biological function. Such a biological function could be scored by various metrics, such as measures of the amount of biochemical that is produced by the cell, or of the activity level of a protein or enzyme produced by the cell, or by some functional property of the cell such as viability, growth rate, or lifespan in certain environments.

Thus, the design problem that arises in synthetic biology is that of designing specific DNA sequences, as well as specifying the supporting environmental and processes state variables-such as type of cell, environmental variables such as temperature, medium composition, concentration of nutrients or chemical stimulating factors provided to the cells in the medium, and process parameters that define a handling or processing procedure that result in achieving a desired outcome of cell function, or of optimizing a property of the cell as assayed by some specific quantitative metric, or of having a list of one or more such metrics, that compose a performance state vector, and optimizing the design so as to have the resulting performance state vector lie in a certain desired region of the state space.

Automation Systems in Synthetic Biology. Because of the extremely high degree of complexity of biological systems, such as a living cell reacting to an environment, there is in practice a highly limited ability to predict the functional properties of a cell from knowledge of its DNA sequence, even though this does in principle define the functional properties of the cell. Therefore, solving a design problem in synthetic biology necessarily requires a large number of empirical trials and testing. As a result, there has generally been a desire to automate the experimental procedures and lab workflows in synthetic biology, such as the primary synthesis the DNA oligomers, the assembly of DNA oligomers into long constructs such as genes or chromosomes, the packaging or transfection of such constructs into cells, and the screening of such transformed cells for function properties. In order to increase the accuracy, precision, consistency and throughout of such procedures, as well as to reduce the time, cost and reagent consumption, it has generally been desirable to transition away from manual lab procedures, towards robotic automation, combined with system miniaturization and microfluidic methods. Many such partially automated systems and processes have been deployed in the context of research and production labs in academia and industry.

In this question for greater automation in synthetic biology, it is especially desirable to map the workflows onto electronic semiconductor chip devices, in order to achieve extremely high degrees of parallelism, miniaturization, and automated control, such as the chip-based devices that have been developed by the patent applicant in, for example, U.S. Pat. No. 12,379,344, which is incorporated herein by reference.

The Use of AI for Predictive Design in Synthetic Biology. Due to the complexity of biological systems, it is very difficult to predict biological function from primary sequence, both at the level of DNA base sequence, and also at the level of protein amino acid sequence. Therefore, there has been great interest in trying to use AI to assist with this prediction of function from sequence, and related abilities to predict the properties of biological systems from the primary state variable s. In general, AI refers to a large and diverse family of computational algorithms which can be used to solve diverse problems that are either difficult for humans to solve, due to their complexity, or otherwise require human-like analytical or pattern recognition skills to solve. One notable example of success in this area is AlphaFold, which predicts the folded structure of a protein from its primary amino acid sequence. AlphaFold uses deep learning neural network algorithms, which were trained on the protein structure data in The Protein Data Bank, which contains hundreds of thousands of folded protein structures that were determined experimentally using X-ray crystallography and other methods. AlphaFold can successfully predict the folded structure of a substantial portion of naturally occurring proteins, with greater accuracy than either human expert predictions, or more classical prediction algorithms, and to within the tolerance of experimental measures in many cases. It is desirable to devise AI methods that can similarly solve other prediction problems, and the related problems of finding a design that will achieve a desired function. More broadly, in the context of synthetic biology, it is desirable to find ways to use AI to assist in solving such design optimization problems.

In this regard, there are presently many efforts underway to use machine learning (“ML”) methods, and related methods such as the more recent generative AI methods, including those based on deep learning neural networks, to produce proposed biological designs that would have desirable functions. It is typically the goal of such methods to generate designs that are intended to have certain desired properties, such as the design of a protein amino acid sequence that would bind—or bind more tightly to—to a target molecule of interest, or the design of an enzyme amino acid sequence that would improve the functions of the enzyme relative to a native form, such as its activity level or stability in a certain environment.

It is assumed there is a given design problem in synthetic biology, where there is a high-level functional goal, such as to design a cell and a related treatment process, so that the cell, undergoing the treatment process, will produce a desired functional result. For example, this function may be the production of a certain chemical, via a biochemical pathway, or production of a protein or antibody or enzyme with certain properties, or a certain cellular phenotype, such as viability, rate of reproduction, or the ability to differentiate into a desired type of cell, or to form organoids (small cell cultures) with certain properties. In general, there will be some performance vector (list) of quantitative or categorical or qualitative functional metrics than can be scored using one or more assays applied to the cell, related to this desired function, and the goal in this context is to achieve this performance vector residing in a desired target region of the that overall state space, or to go as far as possible in certain directions in the state space (i.e., optimize, maximize or minimize various performance parameters). In some preferred embodiments, all such parameters of interest will be represented within a single overall “reward function”, having a numerical value, and the goal is to maximize the reward. More specifically, the design problem will be of the of the form where the goal is to design a DNA construct, such as a gene or set of genes, or a chromosome, and to design the supporting cellular environment, and supporting treatment procedure, to achieve such desired functionality, or to maximize a specific reward function, from the above. In preferred embodiments, this may also be done in a cell-free context, utilizing the well-known methods of cell-free DNA and protein expression, in which the relevant biological processes occur within a droplet, and the biochemical processes within the droplet are to be optimized in this fashion, and there is no biological cell involved (or, conceptually, the droplet plays the role of the “cell” in such cases of cellfree synthetic biology).

The AI disclosed herein is an AI that generally has the capability to take a new set of training data and generate new candidate designs based on the training data (and possibly prior training data that may have been used to pre-train the AI initially).

It is also specifically contemplated that there is a high throughput experimental device, that can take a set of candidate designs, and experimentally produce the required biological constructs, and carry out the specified treatment procedures, and perform the functional scoring assays that constitute the trial results for testing the designs. In preferred embodiments, this experimental platform is based on electronic semiconductor chip devices which carry out the key operations of DNA oligo synthesis, assembly of DNA oligos into the desired DNA payloads, the delivery of these payloads into cells (or cell-free expression droplets), and treatment of these cells according to the specified treatments, and the functional scoring of the results using appropriate assays to obtain the performance scoring vectors.

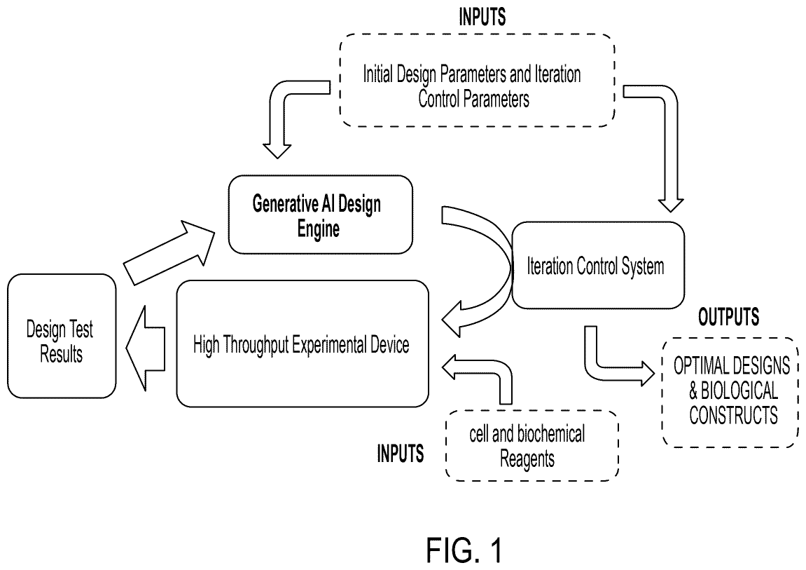

In this context, this disclosure provides that the design optimization problem can be addressed and solved by a method and system, as indicated in FIG. 1, which produces optimal designs (and the biological representative of these designs), by the method disclosed as follows:
(0) An initial set of trial designs is provided. In preferred embodiments, these initial designs may come from the generative AI, or in preferred embodiments they may be otherwise provided. In preferred embodiments, such a first set may be randomly sampled from the possible design space, through any means of random sampling, or random sampling conditioned on prior biological knowledge, such as known naturally occurring DNA or protein sequences, and common naturally occurring cells and conditions, and known cells types and conditions and treatments such as from existing literature or lab procedures), or may similarly be based on such random variations applied to make modifications of an initial set of (perhaps just one or few) candidate designs already provided from prior knowledge, or provided from the generative AI, thereby expanding such a set of “seed” designs into a larger set, as large as is desired.
(1) the experimental system is used to produce a first set of design trial results, by creating and testing the initial set of designs, to produce the performance score vectors for each of the designs.
(2) The generative AI is then trained additionally on the trial results from this initial set, and the is used to generate a second set of candidate designs. This second set may, in preferred embodiments, be further expanded by random variation techniques as noted in (0), applied to produce even more variants from the AI-generated set.
(3) The iteration is repeated by going back to step (1) with this new set of designs. This iterative AI method is repeated until a stopping criterion is achieved. In preferred embodiments, the stopping criteria may be that the performance vector of a subset of the designs achieves the target, or in the case of parameter maximization/minimization or reward maximization, the iteration may stop when either certain target thresholds are reached, or when the rate of increase or decrease of the parameters undergoing optimization slows to a specified threshold.
(4) The resulting output of the system and method is foremost a set of optimal designs, and also (optionally as desired) the actual (live) biological material cell constructs that represent these designs.

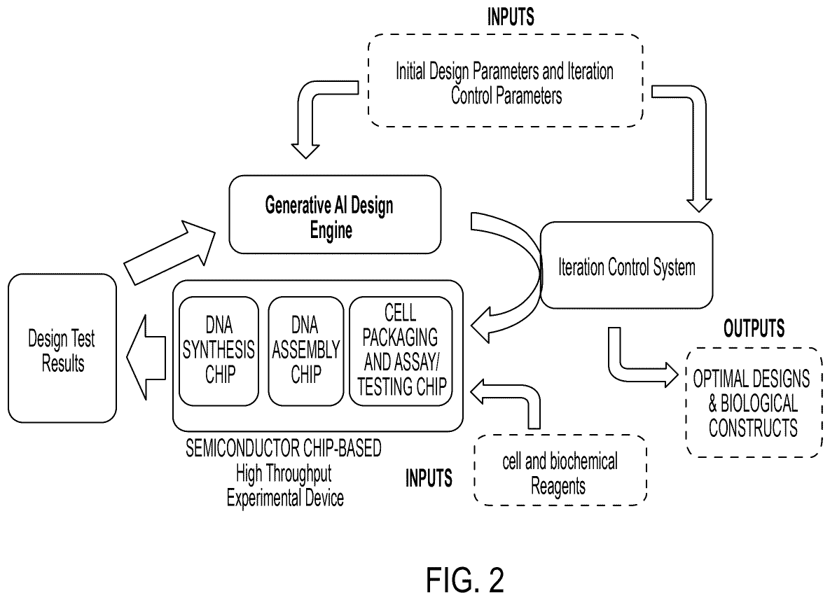

In certain embodiments, as illustrated in FIG. 2, the experimental system is a chip-based system in which highly scalable semiconductor chip devices perform the key workflow processes of DNA oligo synthesis, DNA gene and larger payload assembly, delivery of DNA payloads into cell or cell-free droplets, treatment application, and functional scoring of results. The advantage is that such system automates the end-to-end synthetic biology workflow, in a maximally scalable way, for the greatest advantages in terms of high throughput, low cost, low reagent volume consumption, and precision process control.

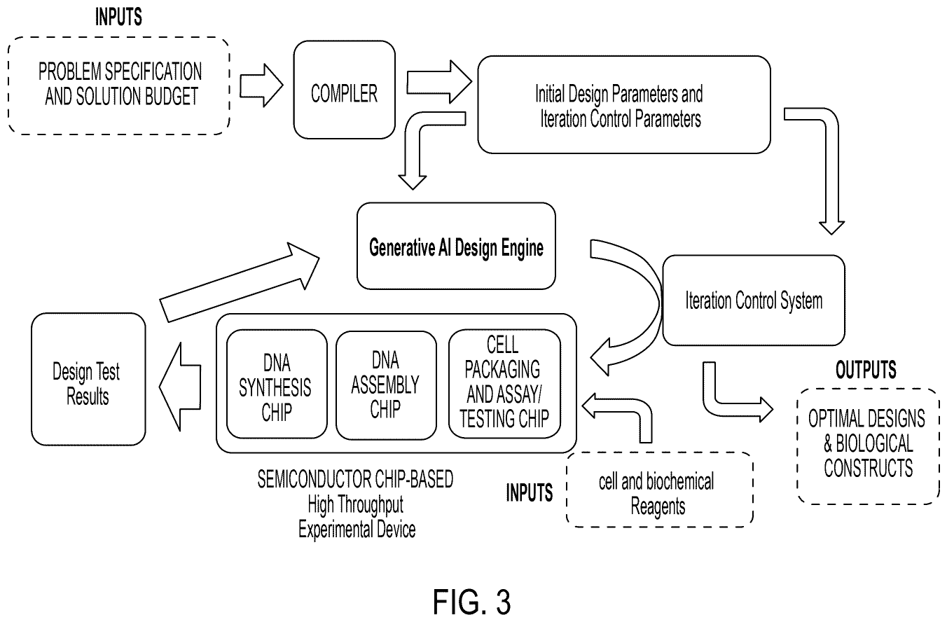

In preferred embodiments, as illustrated in FIG. 3, the iterative method and system can be made into a fully autonomous design optimization system and method, by providing a “problem compiler”, that takes a high-level problem statement, and a solution budget (such as the total number of cycles, or trials, to allow) and compiles that into the initial specification and iteration control parameters for the core system as in FIG. 2. The net result and benefit is that such a system takes a high level problem specification, such as finding a pathway to produce a certain given chemical, or finding an antibody to bind a certain given antigen, or finding an enzyme with certain properties, or creating a cell with a certain phenotype, or an organoid with certain phenotypic properties, and produces in a fully autonomous fashion, an optimized design (and the biological representative of this design).

In certain embodiments, the generative AI that is to be plugged into the method may be one that comprised algorithms such as deep learning, neural nets, generative AI, diffusion methods, decision trees, random forests, and other ML methods that are well known to those skilled in the arts of AI machine learning algorithms. In preferred embodiments, the generative AI may be ones such as RFdiffusion, that generate protein designs, and other such methods that are well known to those skilled in the art of AI for protein engineering, antibody engineering, and other biological applications.

In a preferred embodiment, the central AI can simply comprise a variant generation engine, and a selection method, and perform iterations of variant design generation, scoring of designs, selection, and re-expansion. Such algorithms can be based on specific techniques for sampling and selection from genetic programming, simulated annealing, and Monte Carlo methods, which are known to those skilled in the art. In the present context of synthetic biology design, the variant expansion may expand a given sequence (DNA bases or protein amino acids) into variants based on methods such as random mutation and cross-overs, and conditioned on known sequences and sequence conservation data from known biological sequences and on the redundancy of the genetic code for DNA sequence, and other well-known means of generating biologically conditioned random variation, such that in each iteration, a fraction of the best scoring designs are selected and used as seeds for such variation generation, such that this selected high scoring fraction is re-expanded to a full sized set of designs-in conjunction with a variation generator for the other parameters that control the treatment process-and the process repeats. In preferred embodiments, this type of iteration is combined with genetic programming and/or simulated annealing and/or Monte Carlo optimization methods, which provides addition sampling strategies for the mutation for generating sequence variants from the selected high-scoring pool, as well as the selection strategies for selecting the high-scoring pool of designs. In preferred embodiments, the total variant set may have N members, which N may be up to 1000, 10,000, 100,000, 1 million, 10 million, 100 million, 1 billion or more, and the high performing fraction may be the top 0.01%, top 0.1%, top 1%, top 10% or in the range of the top 0.001% to top 20%. In preferred embodiments, this selection may be based deterministically on the actual top-most scores, or by a simulated annealing or Monte Carlo sampling (probabilistic) selection method to obtain this top set. Such optimization methods that directly fit into this iterative context, such as genetic programming and simulated annealing and others are well-known to those skilled in the art of optimization theory. In this preferred embodiment, there is no need for any other form of AI to assist in this iterative AI method.

In aspects of the present disclosure, a method and system for AI-assisted optimal design in synthetic biology is disclosed. The method and system includes a high-throughput experimental device; a generative AI; a problem specification, experimental design, and scoring assays, in which a first set of designs (which may be given, generated by the AI, or any combination of these, plus optional expansion of such a variation set by random variation methods) tested and scored using the high-throughput experimental device, the generative AI trains on these elements and generates new designs, and the process is iterated until the optimization criteria or termination conditions are achieved.

In embodiments, the high-throughput experimental device is a semiconductor chip-based device.

In embodiments, the method and system is combined with a problem compiler, in which a high-level problem and solution budget are provided, and the problem compiler translates these into the initiation and control parameters of the system, and this iterates until certain stopping criteria are met.

In embodiments, the AI is a generative AI. In embodiments, the generative AI is based on deep learning neural networks or diffusion methods.

In embodiments, the AI algorithm is based on selection of a top performing fraction of designs, combined with random variation expansion of the designs, and using methods selection and variation methods selected from genetic programming algorithms, and/or simulated annealing algorithms, and/or Monte Carlo sampling algorithms.

In embodiments, the method and system is optimized to deliver biological DNA, protein, or cellular living materials representing optimal synthetic biology designs.

In another aspect, a method of optimizing design in synthetic biology by utilizing assistance of AI is disclosed. The method involves receiving a first set of designs, wherein the first set of designs have been tested and scored using a high-throughput experimental device; training a generative AI system using the first set of designs; generating new designs using the generative AI system; and iterating steps of testing and scoring designs; training a generative AI system based on the tested and scored designs; and generating a new set of designs using the generative AI system until at least one of optimization criteria or termination conditions are achieved.

In embodiments, the high-throughput experimental device is a semiconductor chip-based device.

In embodiments, the method involves providing a problem compiler having a high-level problem and a solution budget, and using the program compiler, translating the high-level problem and the solution budget into initiating and control parameters used in the iteration steps, and iterating until stopping criteria are met.

In embodiments, the AI is a generative AI. In embodiments, the generative AI is based on deep learning neural networks or diffusion methods.

In embodiments, the AI algorithm is based on selection of a top performing fraction of designs, combined with random variation expansion of the designs, and using methods selection and variation methods selected from genetic programming algorithms, and/or simulated annealing algorithms, and/or Monte Carlo sampling algorithms.

In embodiments, the method and system is optimized to deliver biological DNA, protein, or cellular living materials representing optimal synthetic biology designs.

### Definitions and Interpretations

As used herein, the terms “AI” or “Artificial Intelligence” refer to an algorithm or collection of algorithms that are used to solve a posed problem. As used herein, the term “generative AI” refers to any AI that generates designs and learns from generated experimental data against those designs, including but not limited to active learning, Boltzmann optimization, deep learning, neural nets, generative AI, diffusion methods, decision trees, random forests, and other ML methods.

As used herein, “DNA” refers to the any form of nucleic acid polymers as makes sense in context for synthetic biology applications, including DNA, its analogues, including epigenetic marks, as well as RNA, and including the range of length from oligos, to genes, to long multi-gene segments, to chromosomes, and, as makes sense in context, collections of DNAs of such lengths. The term “nucleic acid” may also be used to refer to DNA, RNA, or any variants thereof.

As used herein, a “cell” refers to a biological cell, such as may make sense in context, a viral particle, a bacterium, or a eukaryote or plant, insect, fungus, animal, mammalian or human cell. As makes sense in context, “cell” could also represent a small population of cells, up to 10, up to 100, or up to thousands, or a mixture of such populations, or a cultured organoid. As makes sense in context, “cell” could also refer to a droplet in a cell-free expression system or small volume of such solution.

As used herein, a “biological function” or “outcome”, refers to any observable or measurable property of a biological system.

As used herein, a “synthetic biology design” means a specification of all the essential state variables required to specify a biological system that exhibits a certain function on certain circumstance, and as makes sense in context, this may comprise DNA sequences, protein amino acid sequences, cell types to contain the DNA or proteins, organisms, culture media, environmental conditions, and well as a treatment process such as exposure to certain nutrients or chemical signals in a certain order, duration and dose, or growth to a level of multiple cells, cell populations, or organoids, as well as the cell-free expression versions of such things as makes sense in context.

As used herein, “chip” means an electronic integrated circuit chip, and in context could be in particular a CMOS (Complementary Metal Oxide Semiconductor) chip or a TFT (Thin Film Transistor) chip.

What is claimed is:

1. A system for artificial intelligence (AI)-assisted production of an optimal design in synthetic biology, comprising:
a high-throughput experimental device;
a generative AI; and
experimental designs;
wherein a first set of designs of the experimental designs are configured to be tested and scored by the high-throughput experimental device and used for training the generative AI for production of a new set of designs by the generative AI.

2. The system of claim 1, further comprising functional scoring assays configured to constitute trial results for testing the first set of designs.

3. The system of claim 1, further comprising a problem compiler configured to be provided with a high-level problem statement, and a solution budget; and wherein the problem compiler is configured to use the high-level problem statement and the solution budget to compile initial specification and iteration control parameters for the system.

4. The system of claim 1, wherein the production of a new set of designs is iterated until achieving optimization criteria or termination conditions.

5. The system of claim 1, wherein the high-throughput experimental device is a semiconductor chip-based device.

6. The system of claim 5, wherein the semiconductor chip-based device comprises any one or more of a cell packaging and assay testing chip, a DNA assembly chip, and a DNA synthesis chip.

7. The system of claim 1, wherein a biological representative of the optimal design comprises: DNA sequences, protein amino acid sequences, cell types to contain DNA, cell types to contain proteins, organisms, culture media, environmental conditions, treatment processes for exposure to nutrients or chemical signals, and cell-free expressions.

8. The system of claim 7, wherein the treatment processes comprise exposure to nutrients or chemical signals in a certain order, duration and dose, or growth to a level of multiple cells, cell populations, or organoids.

9. The system of claim 1, wherein the generative AI is based on deep learning neural networks, or diffusion methods.

10. The system of claim 1, wherein outputs of the system comprise biological constructs.

11. The system of claim 10, further comprising a delivery system of the biological constructs.

12. A method of determining an optimal design for synthetic biology using generative AI, comprising:
providing initial design parameters and iteration control parameters to a high-throughput experimental device;
providing an initial set of trial designs;
producing a set of design trial results;
training a generative AI system using the set of design trial results; and
generating a new design using the generative AI system.

13. The method of claim 12, further comprising:
further training the generative AI system on the new design; and
producing further sets of new designs; and
wherein the steps of further training and producing further sets of new designs are an iterative AI method.

14. The method of claim 13, wherein the iterative AI method is repeated until a termination condition is achieved.

15. The method of claim 14, wherein termination condition comprises: a performance vector of a subset of produced designs achieving a predetermined target, reaching a target threshold, and a rate of increase or decrease of the initial design parameters and iteration control parameters undergoing optimization reaching a specified threshold.

16. The method of claim 12, wherein the method using generative AI is fully autonomous.

17. The method of claim 12, wherein the high-throughput experimental device is a semiconductor chip-based device.

18. The method of claim 12, further comprising: producing a biological representative of the determined optimal design.

19. The method of claim 18, wherein the biological representative comprises: DNA sequences, protein amino acid sequences, cell types to contain DNA, cell types to contain proteins, organisms, culture media, environmental conditions, treatment processes for exposure to nutrients or chemical signals, and cell-free expressions.

20. The method of claim 12, wherein an algorithm of the generative AI is based on one or more of: selection of a top performing fraction of the design trial results combined with random variation expansion of the design trial results, and methods selection and variation methods from genetic programming algorithms, methods selection and variation methods from simulated annealing algorithms, and methods selection and variation methods from Monte Carlo sampling algorithms.
